(19) United States
(12) Patent Application Publication
(10) Pub. No.: US 2022/0054299 A1
(43) Pub. Date: Feb. 24, 2022

(54) PORTABLE URINARY AID DEVICE

(71) Applicant: SP INTERNATIONAL USA, INC., Whittier (CA)

(72) Inventor: Eui Man YANG, Whittier, CA (US)

(21) Appl. No.: 17/445,587

(22) Filed: Aug. 20, 2021

Related U.S. Application Data
(60) Provisional application No. 63/068,024, filed on Aug. 20, 2020.

(51) Int. Cl. A61F 5/453; A61F 5/455

(52) U.S. Cl. CPC A61F 5/453; A61F 5/4556

(57) ABSTRACT

A portable urinary aid device includes an upper fixture having a ring shape, and a lower fixture configured to be engaged with the upper fixture. The lower fixture has a ring shape to correspond to the upper fixture and has at least one handle extending outwardly from one vertex. When the upper fixture and the lower fixture are assembled by securely engaging with each other, the assembly can secure a plastic bag therebetween.

### INCORPORATION BY REFERENCE TO ANY PRIORITY APPLICATIONS

Any and all applications for which a foreign or domestic priority claim is identified in the Application Data Sheet, or any correction thereto, are hereby incorporated by reference under 37 CFR 1.57. The present application claims the benefit of priority under 35 U.S.C. § 119(e) of U.S. Provisional Patent Application No. 63/068,024, filed on Aug. 20, 2020, and entitled “PORTABLE URINARY AID DEVICE,” which is hereby incorporated herein by reference in its entirety.

### BACKGROUND

### Technical Field

The present disclosure relates to a portable urinary aid device, a method of using thereof, and more particularly, to a portable urinary aid device that allows a user (e.g., a driver, a passenger, a patient, a traveler, etc.) to urinate when a bathroom is not available or not easily accessible.

### Description of the Related Art

Drivers often face a situation in which it is difficult to stop and find a proper place such as a public restroom to relieve an urgent necessity to urinate due to, e.g., heavy traffic or driving on a road where there is no public restroom. In addition, when drivers are in a rush to reach a destination and thus there is inadequate time for the driver to pull off the road and find a public restroom, accidents can happen unintentionally. Moreover, the driver is subjected to discomfort while in this predicament. In a situation where the driver is unable to find a public restroom, the driver may be able to find a relatively convenient location to urinate near the road, but this is sometimes prohibited by law, and it increases the risk of an accident, especially in the dark. Therefore, drivers are often faced with these undesirable situations while on the road.

The above-described situations may also be encountered in other circumstances, for example, while hiking and/or camping, in which a person may not be able to find a nearby available restroom, or a person with restricted mobility may not be able to freely use a restroom even when it is available nearby if there is no assistance available.

### SUMMARY

An aspect of the present disclosure provides a convenient device allowing drivers to relieve themselves while in their vehicles, reducing stress and concern when a long drive is required and avoiding the above-described undesirable situations. The aspect of the present disclosure not only provide convenience in vehicles, but also can be applied to other situations such as camping, hiking, or even at home or a hospital in the event of restricted mobility due to health conditions, post-surgery, etc.

In some aspects, the present disclosure provides a portable urinary aid device comprising: an upper fixture having a ring shape; and a lower fixture configured to be engaged with the upper fixture, the lower fixture having a ring shape to correspond to the upper fixture and having at least one handle extending outwardly from one vertex. When the upper fixture and the lower fixture are engaged, a hollow void is contained therein.

In some aspects, the present disclosure provides a device for securing a urine bag comprising: an upper fixture having a ring shape; a lower fixture configured to be engaged with the upper fixture, the lower fixture having a ring shape to correspond to the upper fixture and having at least one handle coupled to one vertex. When the upper fixture and the lower fixture are assembled, the assembly is configured to secure the urine bag between the upper and lower fixtures.

In some aspects, the present disclosure provides a method of using the portable urinary aid device comprising: detaching the upper fixture from the lower fixture; unlocking a locking element of a urine bag to open an entrance of the urine bag; placing the urine bag and folding over around the lower fixture; and placing the upper fixture on the lower fixture to assemble the upper and lower fixtures.

### BRIEF DESCRIPTION OF THE DRAWINGS

The present disclosure will be further explained with reference to the appended figures which are intended to illustrate and not to limit the present disclosure, wherein like structure is referred to by like numerals throughout the several views.

FIGS. 1A and 1B show examples of urine emergency toilets;

FIGS. 2A, 2B, and 2C show a portable urinary aid device according to an embodiment of the present disclosure, in which FIG. 2A shows a side view of the embodiment, FIG. 2B shows a top view of the embodiment, and FIG. 2C shows a bottom view of the embodiment;

FIGS. 3A and 3B show an upper fixture (also referred to herein as a cap or a top portion) of the portable urinary aid device, in which FIG. 3A shows a top view of the upper fixture and FIG. 3B shows a bottom view of the upper fixture;

FIGS. 4A and 4B show perspective views of a lower fixture (also referred to herein as a body or a bottom portion) of the portable urinary aid device according to an embodiment of the present disclosure;

FIGS. 5A and 5B show the upper fixture and the lower fixture according to an embodiment of the present disclosure in a disassembled view;

FIGS. 6A and 6B shows side views of a urine bag according to an embodiment of the present disclosure;

FIGS. 7A-7D illustrate embodiments in which the urine bag is being secured to the device;

FIG. 8 is a perspective view of a portable urinary aid device having a cap and a body that are assembled with each other;

FIG. 9 is another perspective view of the portable urinary aid device, showing the portable urinary aid device viewed in a viewing direction different from that of FIG. 8;

FIG. 10 is a front elevation view of the portable urinary aid device;

FIG. 11 is a rear elevation view of the portable urinary aid device;

FIG. 12 is a left side elevation view of the portable urinary aid device;

FIG. 13 is a right side elevation view of the portable urinary aid device;

FIG. 14 is a top plan view of the portable urinary aid device;

FIG. 15 is a bottom plan view of the portable urinary aid device;

FIG. 16 is an exploded, perspective view of the portable urinary aid device, showing the cap and the body in a disassembled state;

FIG. 17 is another exploded, perspective view of the portable urinary aid device, showing the cap and the body in a disassembled state, viewed in a viewing direction different from that of FIG. 16;

FIG. 18 is an exploded, front elevation view of the portable urinary aid device;

FIG. 19 is an exploded, rear elevation view of the portable urinary aid device;

FIG. 20 is an exploded, left side elevation view of the portable urinary aid device;

FIG. 21 is an exploded, right side elevation view of the portable urinary aid device;

FIG. 22 is an exploded, top plan view of the portable urinary aid device, showing the cap and the body in a disassembled state;

FIG. 23 is an exploded, bottom plan view of the portable urinary aid device, showing the cap and the body in a disassembled state, viewed in a viewing direction different from that of FIG. 22;

FIG. 24 is a perspective view of the cap;

FIG. 25 is another perspective view of the cap, showing the cap viewed in a viewing direction different from that of FIG. 24;

FIG. 26 is a front elevation view of the cap;

FIG. 27 is a rear elevation view of the cap;

FIG. 28 is a left side elevation view of the cap;

FIG. 29 is a right side elevation view of the cap;

FIG. 30 is a top plan view of the cap;

FIG. 31 is a bottom plan view of the cap;

FIG. 32 is a perspective view of the body;

FIG. 33 is another perspective view of the body, showing the body viewed in a viewing direction different from that of FIG. 32;

FIG. 34 is a front elevation view of the body;

FIG. 35 is a rear elevation view of the body;

FIG. 36 is a left side elevation view of the body;

FIG. 37 is a right side elevation view of the body;

FIG. 38 is a top plan view of the body;

FIG. 39 is a bottom plan view of the body;

FIG. 40 is a section view of the cap taken along a line 33-33 shown in FIG. 30; and

FIG. 41 is a section view of the body taken along a line 33-33 shown in FIG. 38.

### DETAILED DESCRIPTION OF THE EMBODIMENT

Although the disclosure hereof is detailed and exact to enable those skilled in the art to practice embodiments of the invention, the embodiments disclosed herein are merely examples of the disclosure, which may be embodied in other specific structure. While various embodiments have been described, the details may be changed without departing from the disclosure, which is defined by the claims.

As used in this application and in the claims, the singular forms “a,” “an,” and “the” include the plural forms unless the context clearly dictates otherwise. Additionally, the term “includes” means “comprises.” Although the operations of embodiments of the disclosed method may be described in a particular, sequential order for convenient presentation, it should be understood that the disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed herein. Moreover, for the sake of simplicity, the attached figures may not show all the various ways in which the disclosed system, method, and apparatus can be used in combination with other systems, methods, and apparatuses.

### Example Device #1

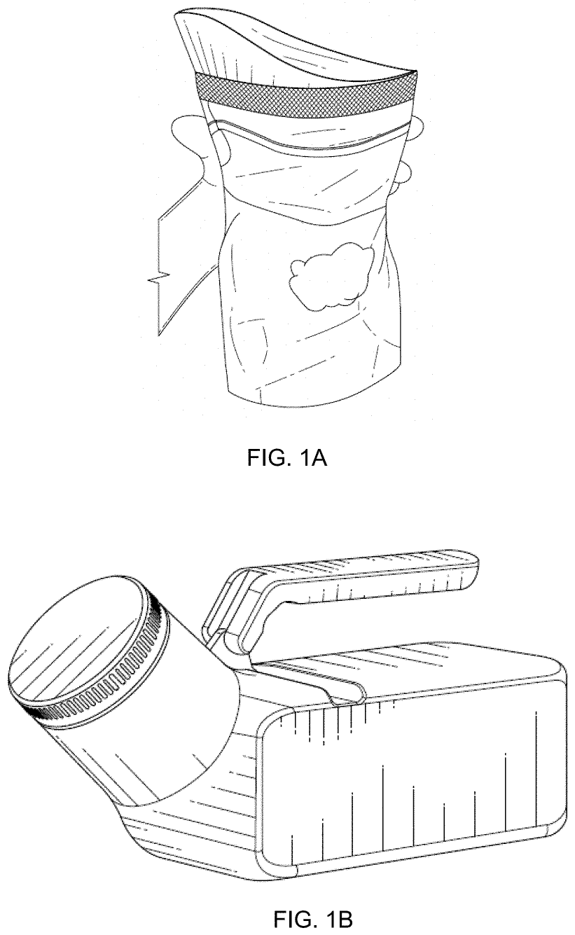

Referring to FIG. 1A illustrating one example, a disposable urine bag is described. The illustrated bag does not have a handle and thus there is a risk of the user may drop the bag before, during, or after use. Moreover, these bags are usually small, so the user may and meant to be held in one hand, so the user would need to support the weight of the urine with his or her hand while holding the bag, which may cause the user to drop the bag or cause the bag to be moved, resulting in spillage. In addition, its upright shape of the bag generally requires the user to be standing while urinating into the bag. Thus, a driver driving in a vehicle or a person with restricted mobility may find such bags inconvenient to use.

Further, some of these urine bags may be equipped with chemical substances such as superabsorbent polymer (SAP) that can absorb urine, vomit, or other liquid waste and turns it into odorless, spill-proof gel. The bags containing special chemicals may not be permitted to be disposed in a general waste bin. These bags also require additional materials increasing the cost associated with manufacturing or using the device on a per-use basis, as these single-use devices incorporate a structural component to provide stability to the bag and/or the opening of the bag during the urination process.

### Example Device #2

FIG. 1B illustrates an example of a portable urinary device made of plastic materials. These larger containers are quite bulky and take up a lot of space, so it may be difficult to store them or carry them around (e.g., a hiker may not have room for it in his or her backpack). Since such a device is not meant for one-time use, after use, the user would need to carry the device around or store the device somewhere until the device can be emptied and cleaned, which may be inconvenient to the user.

### Example Device #3

In another example, a mobile sanitary urinal system (not shown) for truck drivers or others who do not have access to restroom facilities while traveling may include a removable accumulation vessel mounted to an interior or exterior of the motor vehicle. Such a mobile sanitary urinal system may include a catheter-type receiver thereby functioning as a urinal to receive urine from the user and to direct it through the hose to a retention container, which can be manually emptied and cleaned. Such a mobile sanitary urinal system may require a precise coordination by the user since the catheter-type receiver has a small entrance and may be more cumbersome to the user since such a system requires manual emptying and cleaning.

### Technical Advantages

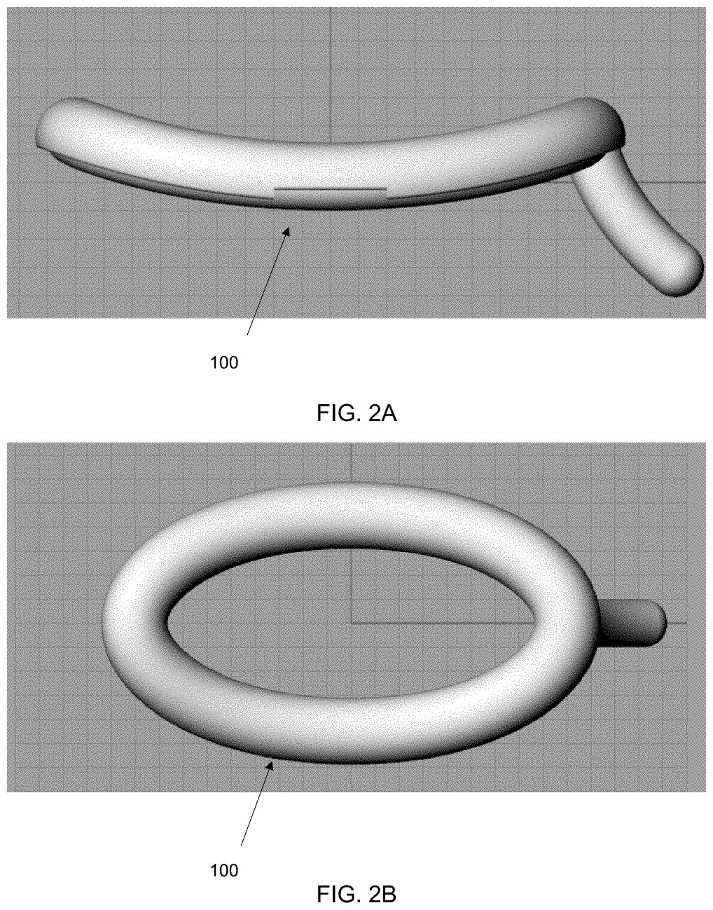

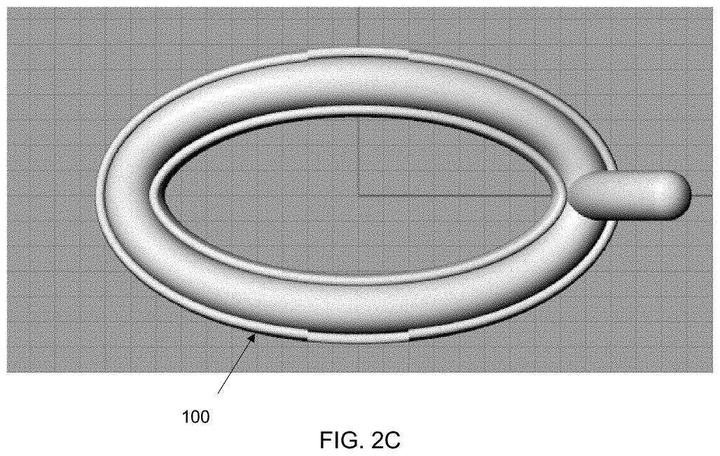

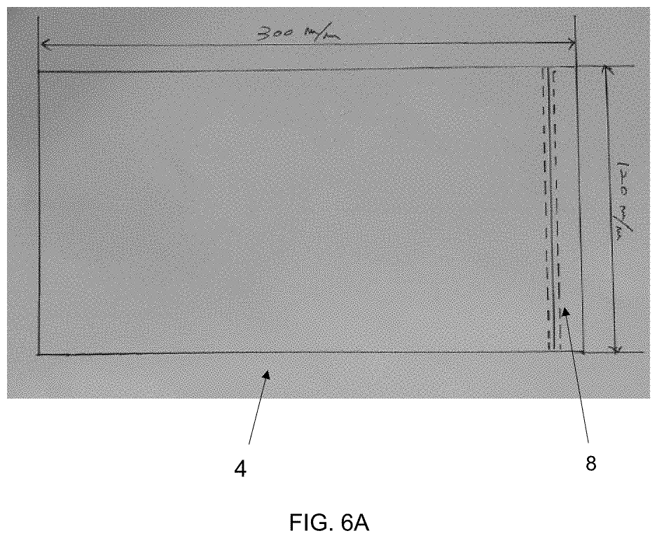

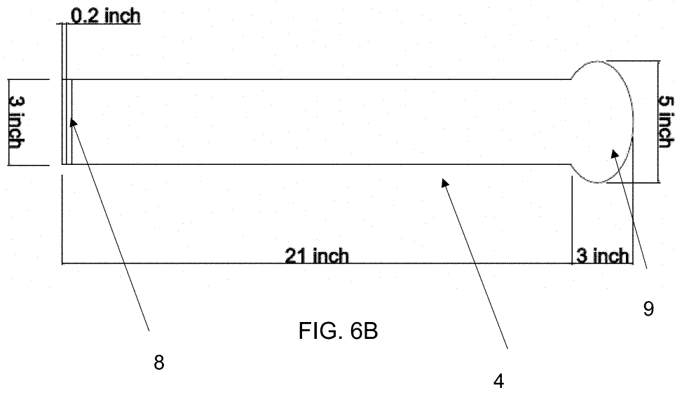

Disclosed embodiments of a portable urinary aid device or system (which may refer to both the physical re-usable piece (e.g., shown in FIG. 2A) and the disposable bag (e.g., shown in FIG. 6B), or refer just to the re-usable piece, depending on the context) can advantageously provide the stability that comes with having a physical handle usually found on a re-usable urinary aid device such as the device shown in FIG. 1B while also providing the sanitary benefits (no need to clean re-usable parts), disposability (bag can be sealed and discarded after use), and portability (takes up a lot less space than bulkier re-usable devices) usually found in disposable urinary aid devices such as the disposable bag shown in FIG. 1A. The disposable bag attachment mechanism provided by the reusable portable urinary aid device allows the users of the device according to the present disclosure to enjoy the advantages of both the reusable portable urinals and the disposable urine bags, which reducing or eliminating one or more of the inconveniences or difficulties described above.

Additionally, the portable urinary aid device according to embodiments described herein can provide another advantage not found in the devices shown in FIGS. 1A and 1B, which is to eliminate the need for the user to be supporting the weight of the bag and/or its content while using the device, by providing a disposable bag that is long enough to touch the ground while in use (e.g., in a car, in a chair, in a bed, etc.).

In addition, the portable urinary aid device as disclosed herein can be manufactured with a simplified design having an integrated portion and thus reducing the cost. Further, the device can have a tube shape having a hollow cavity therein thus further reducing the material cost and reducing the overall weight.

### Technical Details

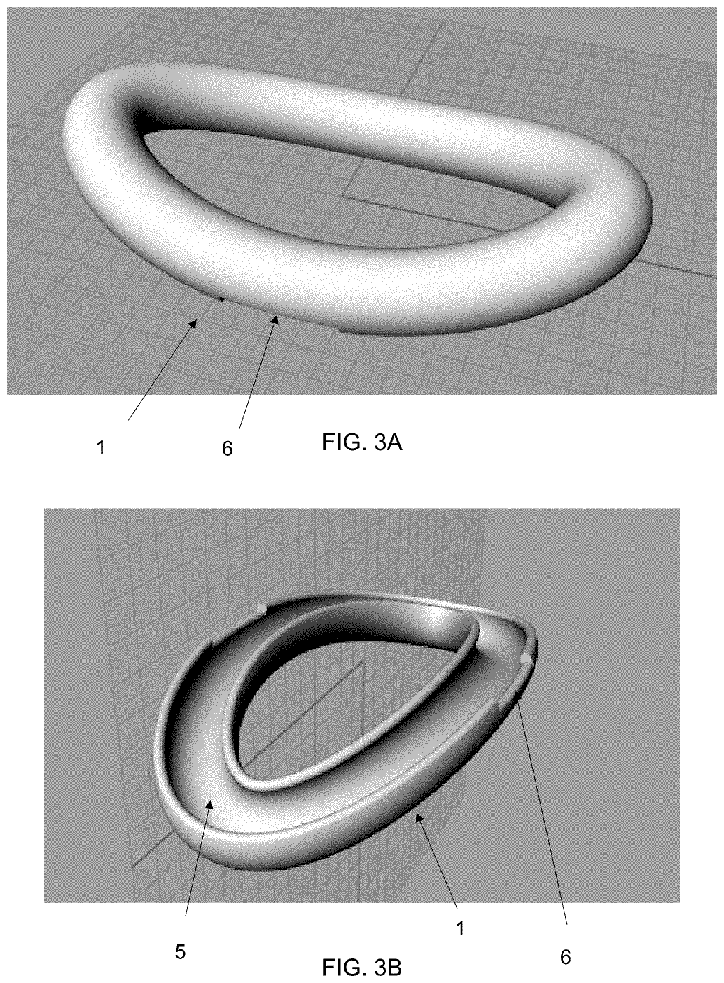

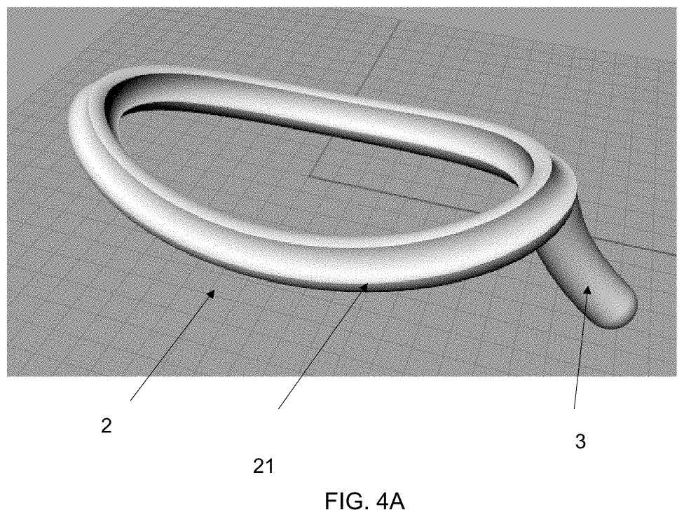

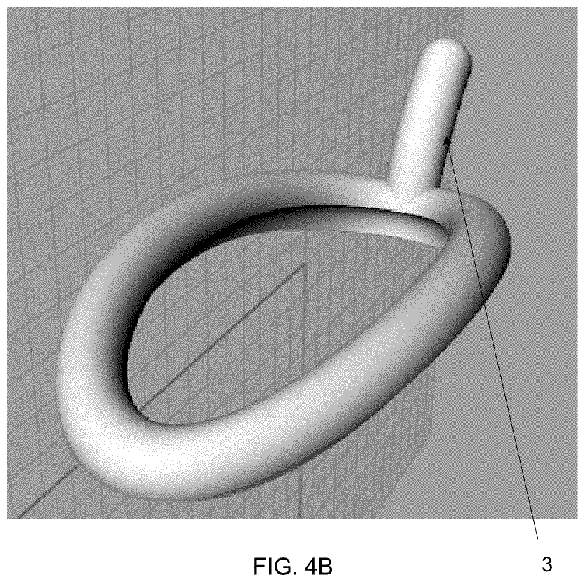

FIGS. 2A, 2B, and 2C respectively illustrate a side view, a top view, and a bottom view of a portable urinary aid device 100 as assembled according to an embodiment of the present disclosure. The portable urinary aid device 100 can include one or more materials. For example, the portable urinary aid device 100 can include an upper fixture or cap 1 (see FIG. 3A) and a lower fixture or body 2 (see FIG. 4A) each may be composed of the same material or different materials. The portable urinary aid device 100 can be made from any suitable material, including, for example, plastic, acrylic, silicone, or the like, or a combination of materials.

When the portable urinary aid device 100 is assembled, in embodiments, the middle portion thereof can be bent downward forming a curvature when viewed from the side (e.g., FIG. 2A). This curvature may make it easier for the user to position the device 100 against the user's body or body part prior to use and to urinate into the bag along the direction of the curvature (e.g., towards the bottom in FIG. 2A). The curvature may especially be convenient if the user is using the device while sitting down (e.g., kid in the back seat, patient in a wheelchair, etc.), as the user can simply point the opposite end from the handle (e.g., left end in FIG. 2A) into the seat and use the curvature to make sure the urine ends up inside the bag.

More specifically, the vertices of the portable urinary aid device 100 may bent upwards for about, e.g., 15° with respect to the co-vertices thereof. The bending degree of 15° is merely an example, and thus, it can be any other ergonomic value. In various embodiments, the bending degree of the upper fixture and the lower fixture corresponds to each other. In addition, the thickness of the portable urinary aid device 100 may be 15 mm when assembled, however it is not limited thereto such that the thickness could be, e.g., 12 mm or any other suitable value.

The assembly may be formed in a ring shape, more particularly in an oval ring shape so as to form a through hole in the center for inserting a urine bag therethrough. The urine bag is described in greater detail below.

Referring to FIGS. 3A and 3B, in some embodiments, the portable urinary aid device 100 may include the upper fixture 1 as noted above. The upper fixture 1 has a smooth upper surface while a lower surface has edges bent upwardly as viewed in FIG. 3B or downwardly when viewed in the assembly (e.g., FIG. 2A), creating a U-shape cross section. This peripheral feature further allows the secure engagement with the lower fixture 2 for tightly holding the urine bag therebetween.

The upper fixture 1 may further include at least two dent portions 6 formed on the outer edge of the upper fixture 1, which allows more convenient disassembly of the upper fixture 1 and the lower fixture 2 to discard the urine bag after the use of the portable urinary aid device (e.g., urination) is completed. In other embodiments, the dent portions are not provided.

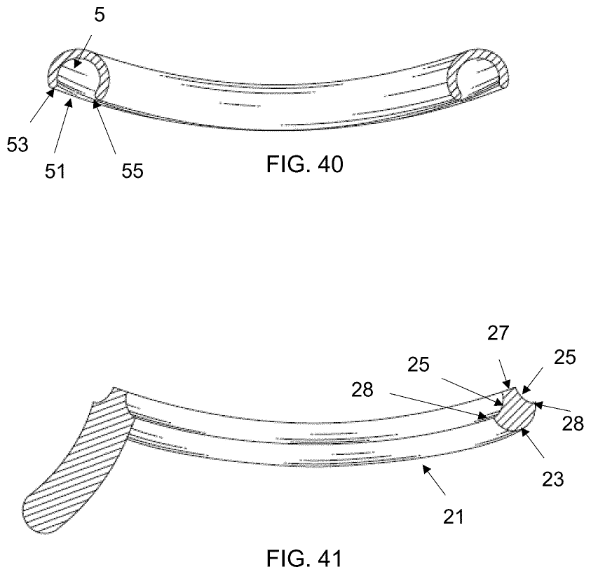

As shown in FIGS. 3B, 5B, 17, 23, 25, 28, 29, 31, and 40, in embodiments, the upper fixture 1 can include a channel 5 extending in a circumferential direction that can receive a rim of the lower fixture 2. The channel 5 has an opening 51 extending along the circumferential direction formed between an inner edge 55 and an outer edge 53 of a lower surface of the lower fixture 1. (See FIG. 40.) Through the opening, the rim of the lower fixture 2 is inserted in the channel or pulled out from the channel. In a section of the upper fixture 1 as shown in FIG. 40, the upper fixture 1 has a round surface to form the channel 5.

In some embodiments, the lower fixture 2 can include a rim 21 and a handle portion 3 attached to the rim 21. The handle 3 diagonally extends out from a lower surface of the rim of the lower fixture 2. As shown in FIGS. 4A and 4B, the handle portion 3 may be curved to improve ergonomic grip such that when a user (e.g., a driver) is using the device of the embodiment, e.g., urinating, the user can comfortably grip the handle to hold the device by one hand (e.g., the user may have pre-assembled the device such that the disposable bag is already attached to the body of the device). The handle portion 3 may have a length that a standard male user can easily and comfortable grip. In another embodiment, the ring shape of the device 100 may be larger than (or the same as) that shown in FIG. 4A and/or the handle may be smaller than (or the same as) that shown in FIG. 4A (e.g., a device usable to female users).

Referring to FIGS. 4A, 16, 18-21, 32, and 34-37 and further referring to FIG. 41 illustrating a section of the rim 21, in the section, the rim 21 includes a round bottom portion 23, an upper stem portion 27 and two recessed portions 25. (See FIG. 41.) When the rim 21 is inserted in the channel 5 of the upper fixture 1, the round bottom portion 23 is exposed through the opening of the channel.

In some embodiments, an upper portion of the lower fixture 2 may have a similar configuration to the channel 5 of the upper fixture 1 for the secure engagement therebetween. For examples, a top surface of the stem portion of the lower fixture 2 may have a round surface as shown in FIG. 4A that is compatible with the round inner surface of the upper fixture 1.

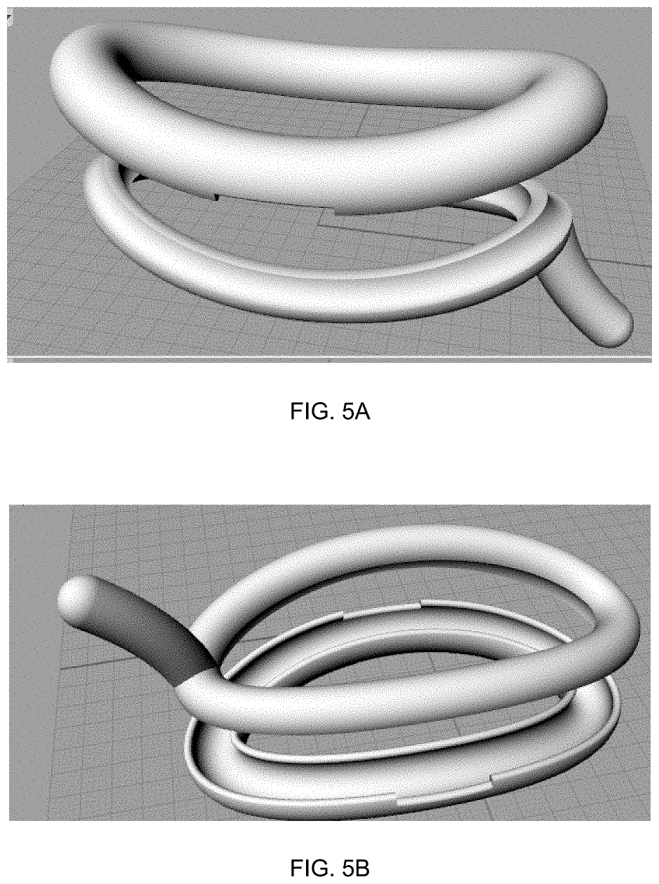

FIGS. 5A and 5B illustrate disassembled views of the upper fixture 1 and the lower fixture 2 to help describe the engagement therebetween. As shown, the rim 21 of the lower fixture 2 engages with the channel 5 of the upper fixture 1. When assembling the upper fixture 1 and the lower fixture 2, the rim 31 of the lower fixture 2 is inserted in the channel 5 through the opening 51. In embodiments, a distance between the edges 53 and 55 of the upper fixture 1 can designed to be smaller than a width or diameter of the rim 23 (e.g., a distance between two edges 28). Thus, the rim 23 is inserted in the channel 5 by a user's force and firmly retained in the channel 5. When inserting the rim 21, the upper fixture 1 can be elastically deformed to receive the rim through the opening and restored after insertion of the rim 21.

In an assembled state, in embodiments, the edges 28 of the lower fixture 2 are in the channel 5 and the edges 53 and 55 of the upper fixture 1 are placed over the round bottom portion 23 so that the upper fixture can retain the rim 21 of the lower fixture 2 (see, e.g., FIGS. 2C and 9). Thus, the two fixtures 1, 2 can be securely engaged with each other without any additional component. Then, as noted above, the at least two dent portions 6 allow the assembly of the device can be easily disassembled by a user.

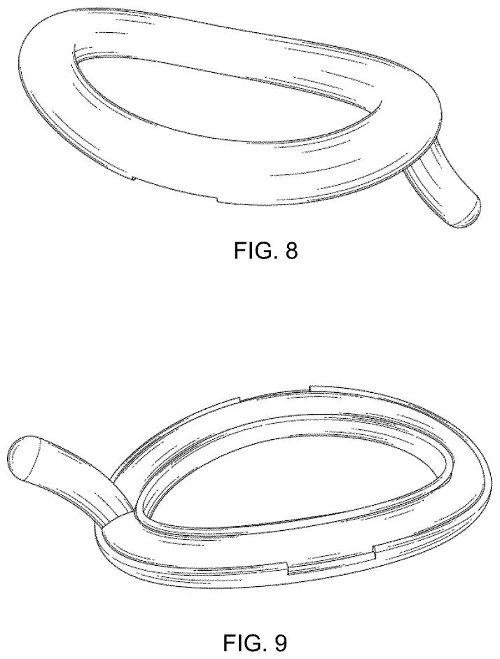

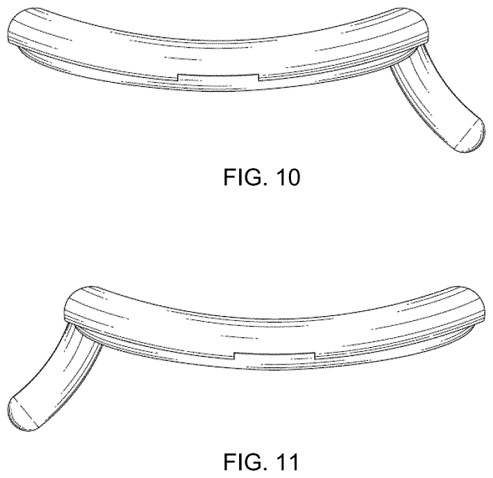

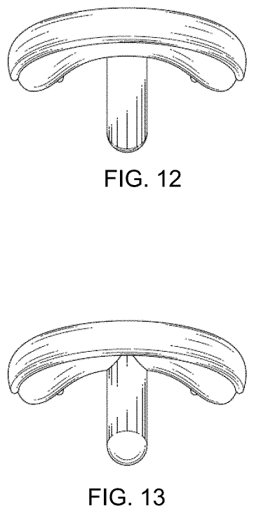

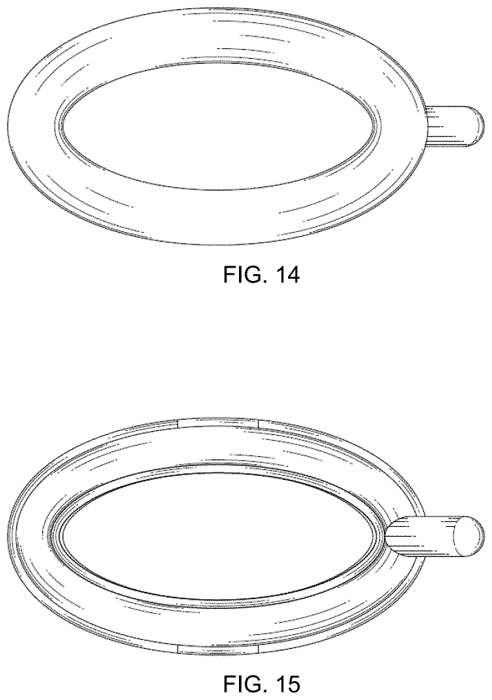

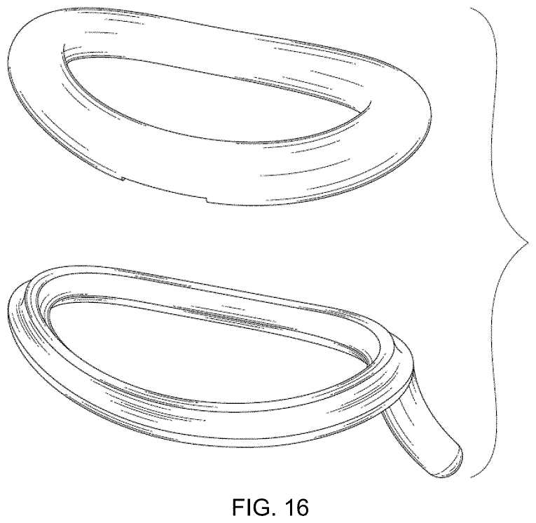

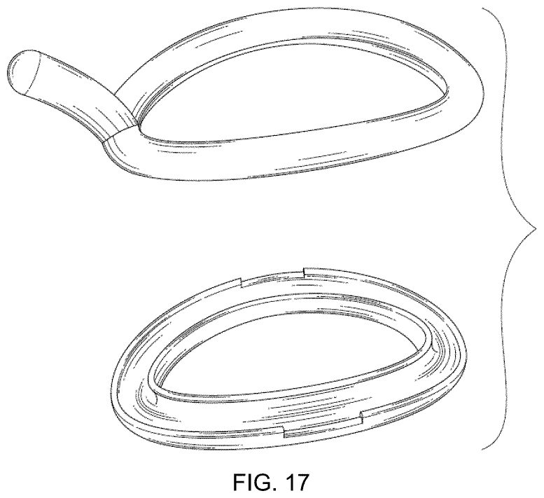

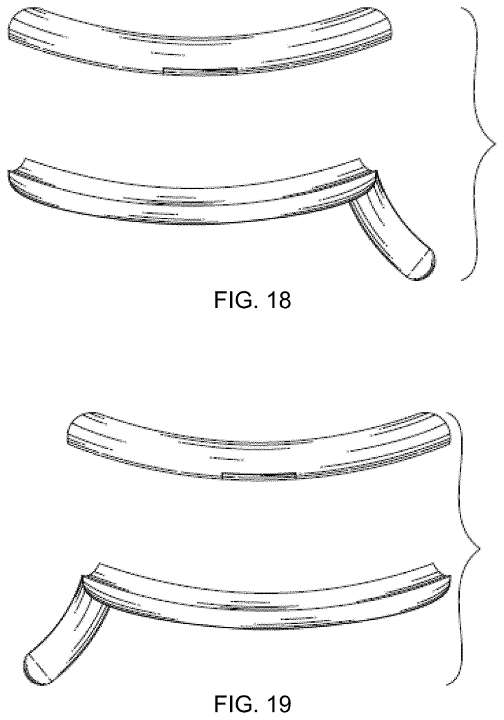

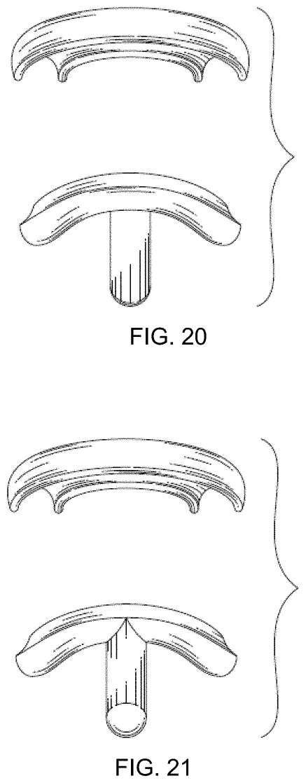

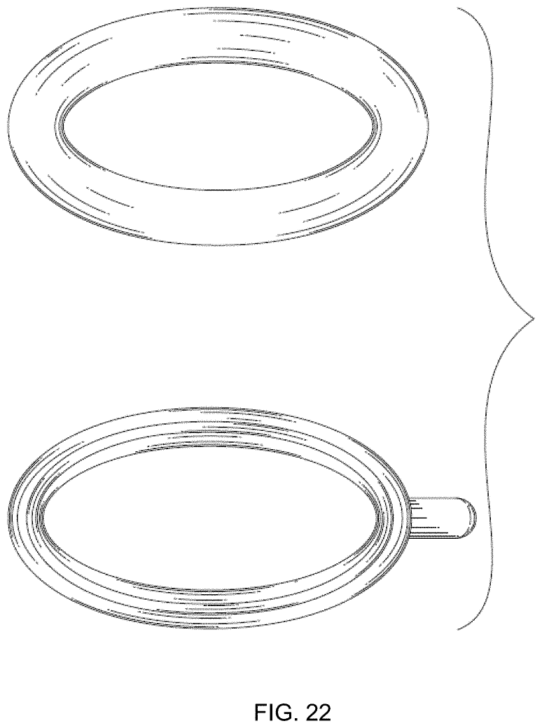

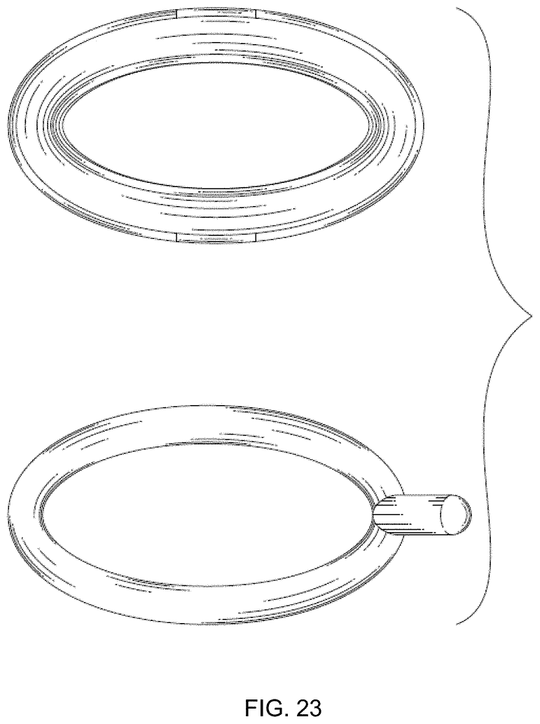

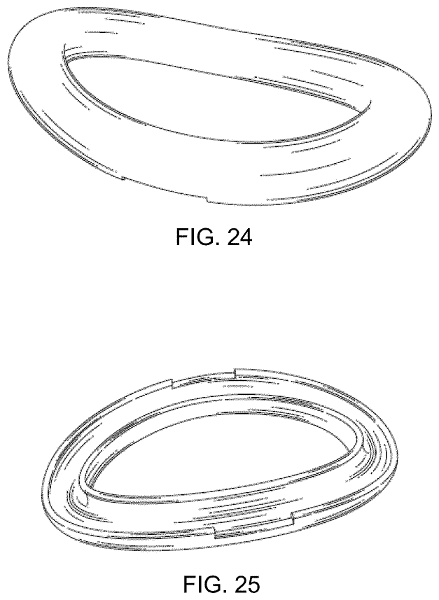

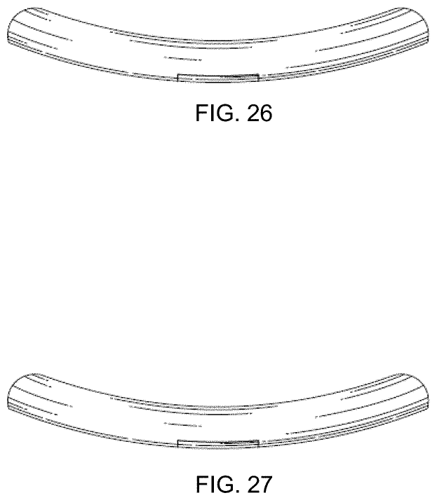

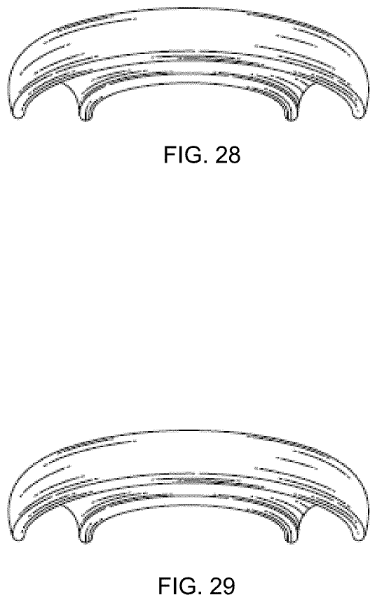

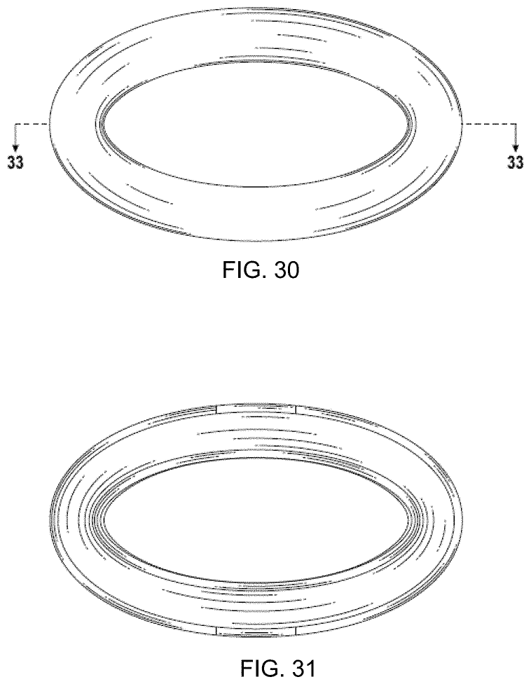

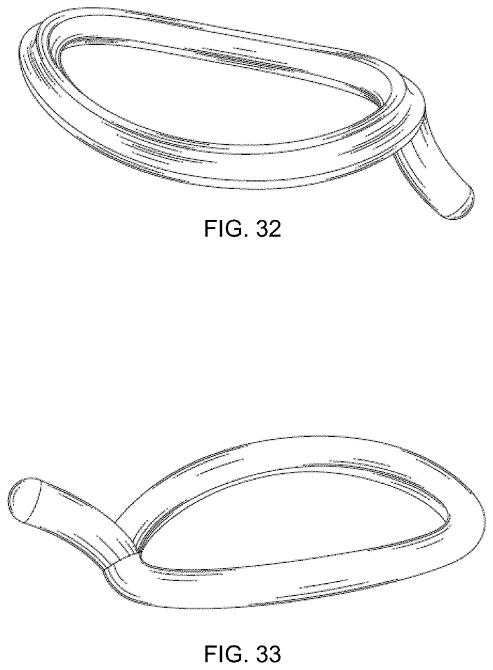

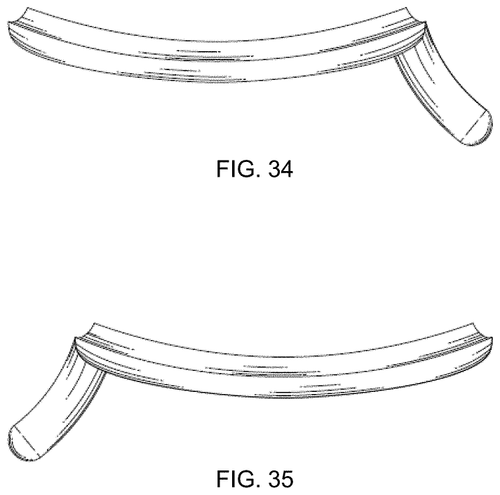

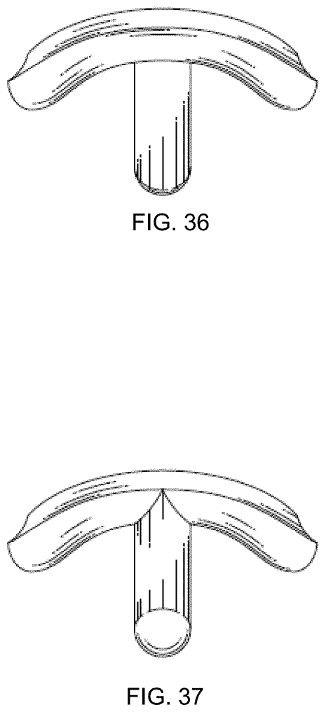

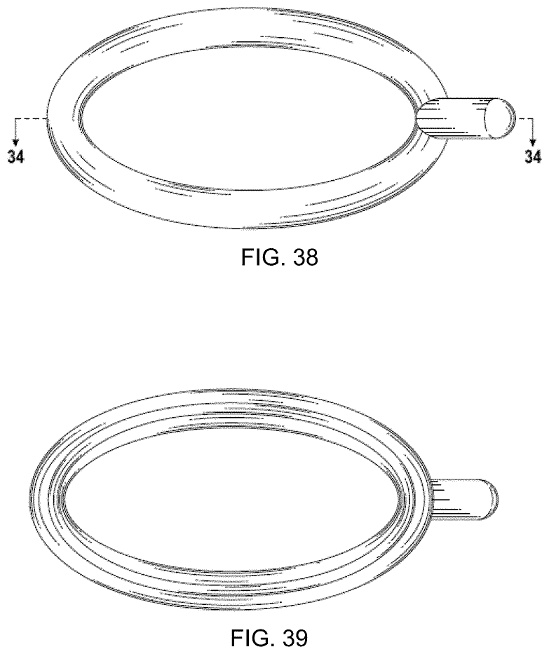

Additional views of the portable urinary aid device are described in FIGS. 8-42. For example, FIG. 8 is a perspective view of a portable urinary aid device having a cap and a body that are assembled with each other; FIG. 9 is another perspective view of the portable urinary aid device, showing the portable urinary aid device viewed in a viewing direction different from that of FIG. 8; FIG. 10 is a front elevation view of the portable urinary aid device; FIG. 11 is a rear elevation view of the portable urinary aid device; FIG. 12 is a left side elevation view of the portable urinary aid device; FIG. 13 is a right side elevation view of the portable urinary aid device; FIG. 14 is a top plan view of the portable urinary aid device; FIG. 15 is a bottom plan view of the portable urinary aid device; FIG. 16 is an exploded, perspective view of the portable urinary aid device, showing the cap and the body in a disassembled state; FIG. 17 is another exploded, perspective view of the portable urinary aid device, showing the cap and the body in a disassembled state, viewed in a viewing direction different from that of FIG. 16; FIG. 18 is an exploded, front elevation view of the portable urinary aid device; FIG. 19 is an exploded, rear elevation view of the portable urinary aid device; FIG. 20 is an exploded, left side elevation view of the portable urinary aid device; FIG. 21 is an exploded, right side elevation view of the portable urinary aid device; FIG. 22 is an exploded, top plan view of the portable urinary aid device, showing the cap and the body in a disassembled state; FIG. 23 is an exploded, bottom plan view of the portable urinary aid device, showing the cap and the body in a disassembled state, viewed in a viewing direction different from that of FIG. 22; FIG. 24 is a perspective view of the cap; FIG. 25 is another perspective view of the cap, showing the cap viewed in a viewing direction different from that of FIG. 24; FIG. 26 is a front elevation view of the cap; FIG. 27 is a rear elevation view of the cap; FIG. 28 is a left side elevation view of the cap; FIG. 29 is a right side elevation view of the cap; FIG. 30 is a top plan view of the cap; FIG. 31 is a bottom plan view of the cap; FIG. 32 is a perspective view of the body; FIG. 33 is another perspective view of the body, showing the body viewed in a viewing direction different from that of FIG. 32; FIG. 34 is a front elevation view of the body; FIG. 35 is a rear elevation view of the body; FIG. 36 is a left side elevation view of the body; FIG. 37 is a right side elevation view of the body; FIG. 38 is a top plan view of the body; FIG. 39 is a bottom plan view of the body; FIG. 40 is a bottom plan view of the body; FIG. 41 is a section view of the cap taken along a line 33-33 shown in FIG. 30; and FIG. 42 is a section view of the body taken along a line 33-33 shown in FIG. 38.

In various embodiments, the urine bag 4 may be formed with a plastic zipper (element 8 in FIGS. 6A and 6B) to securely enclose the contained content (e.g., urine) after use of the bag. The plastic zipper 8 may be a sliding plastic zipper or a slideless plastic zipper. In other embodiments, the urine bag 4 may be sealed with a zip tie or with an adhesive. The urine bag 4 can be made of any durable material, e.g., a rubber material, polyethylene, or the like. FIGS. 6A and 6B illustrate examples of bags that can be used as urine bags for the portable urinary aid device of the present embodiment, while the precise dimensions thereof are for illustration purposes only, and may vary in practice. As shown in FIG. 6A, the urine bag 4 may have a locking element 8 such as a zipper as described above at one end of the urine bag 4 as an entrance side. As shown in FIG. 6B, the distal end of the urine bag 4 may have a larger volume as a storage portion 9 to securely contain urine, and the urine bag 4 may have a length sufficient to extend to the lower interior surface of the vehicle when a user, e.g., a driver or a passenger is sitting down while using the device 100.

In some embodiments, the urine bag 4 may be made of plastic, e.g., polyvinyl, but the urine bag 4 is not limited thereto, and may be made of, or further include, acrylic, silicone, woven or non-woven fabric, paper with a waterproof coating, or the like, or a combination of materials.

The urine bag 4 is however not limited to the above description, and any suitable flexible container having a size to fit over the lower fixture 2 can be used in substitution for the urine bag 4 of the present embodiment described above.

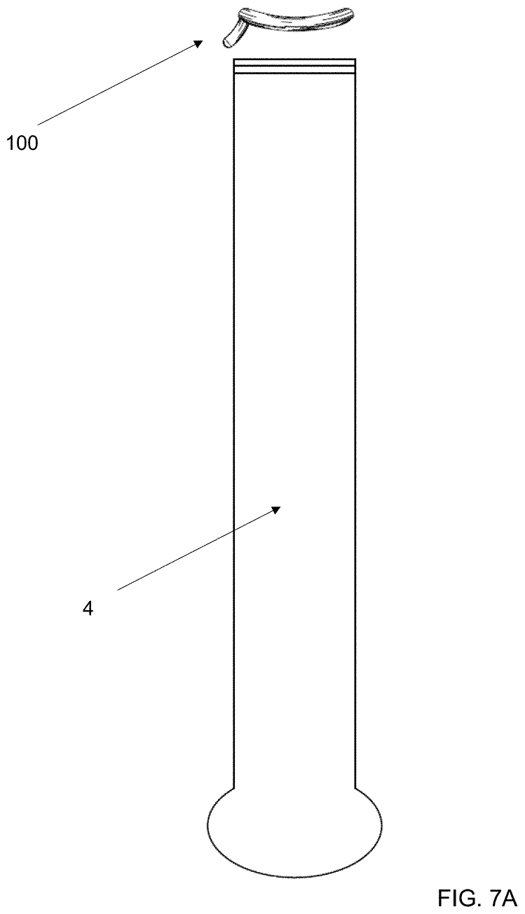

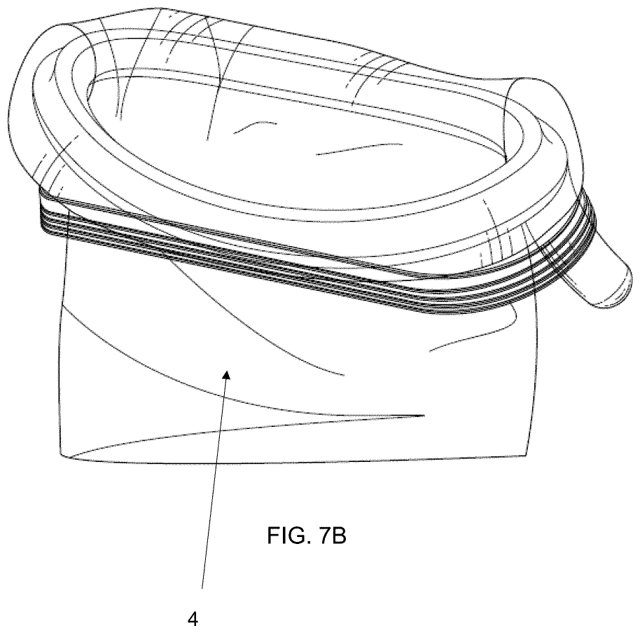

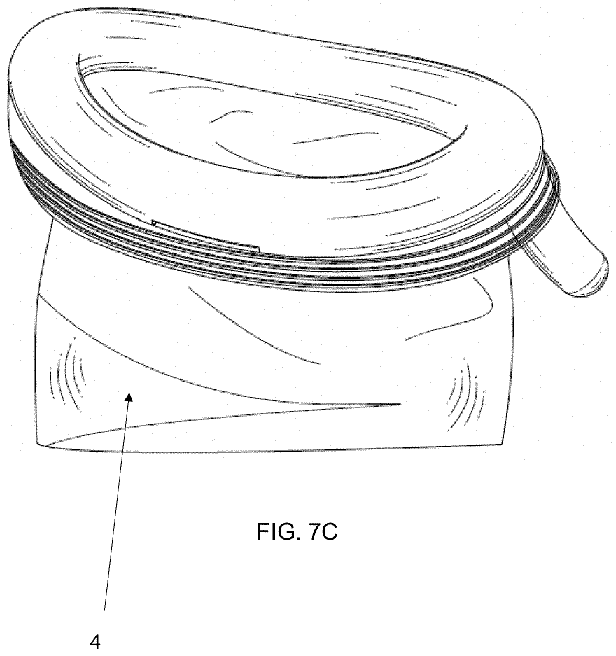

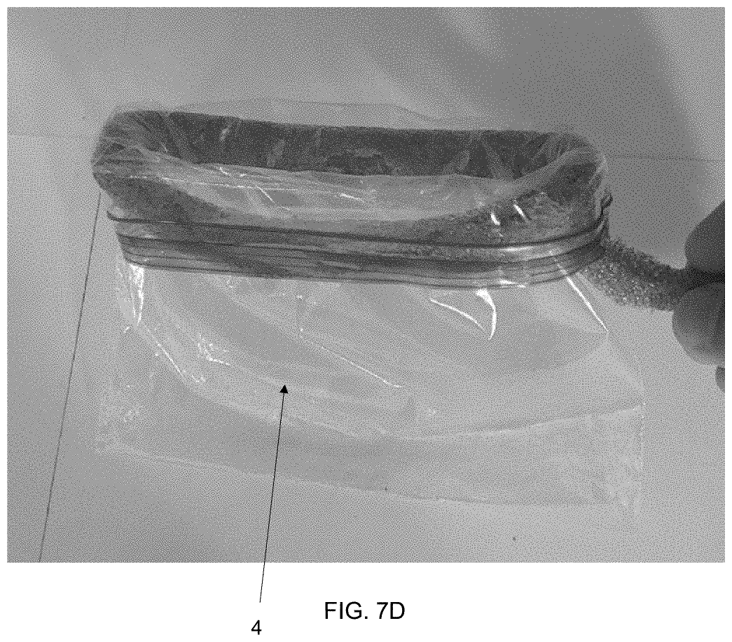

Referring to FIG. 7A, when installing the bag 4, a lip portion of the opening of the bag 4 where the zipper 8 is located is inserted in a center hole of the rim 21 of the lower fixture 2. Thereafter, the lip portion of the bag 4 is bent throughout the circumference of the lip portion to wrap around the rim 21 as shown in FIG. 7B illustrating an example. Subsequently, the rim 21 of the low fixture 2 is inserted in the channel 5 of the upper fixture 1. In this assembled state, the lip portion of the bag is snared between the upper fixture 1 and the lower fixture 2. In embodiments, the zipper portion of the bag 4 can be positioned between the inner surface of the channel 5 and the recessed portion 25 of the rim 21, but not limited thereto.

As shown in FIG. 7B, a urine bag 4 can be disposed to cover the upper surface of the lower fixture 2 and extend downwards to provide a receptacle for urine. In certain embodiments, when the fixtures 1, 2 are disassembled, the urine bag 4 can be secured on top of the lower fixture 2 first and then the upper fixture 1 can be engaged to secure the urine bag therebetween, as shown in FIG. 7C. In this way, even when the urine bag 4 is relatively long and touches the vehicle floor, the device of the present embodiment can prevent any leakage from the upper opening, or the urine bag from unexpectedly detaching from the device. The urine bag 4 may be a disposable bag, a plastic bag, or a plastic disposable bag.

In some embodiments, the bag 4 can go over the entire device 100 as shown in FIG. 7D. In such embodiments, the device 100 may be a single-piece device (instead of two-piece device as shown in FIG. 5A) and once the bag 4 wrapped around the device 100, the user may use the device 100 (e.g., without attaching a cap or another piece thereto). In such embodiments, the bag may be made from material that would not allow the bag to easily slip off the ring portion of the device 100. Additionally or alternatively, the bag and/or the ring portion of the device may include adhesive materials that allows the bag, once in contact with or wrapped around the ring portion, to stay on the ring portion without being detached from the ring portion. Although several disposable-bag-attachment mechanisms are described herein (e.g., top and bottom portions of the device 100 holding the bag in place, non-slip material, adhesive, etc.), other techniques for attaching disposable bags to the reusable device 100 may be used (e.g., bag being clipped to the device 100, bag being held by magnetic force, etc.).

Although FIGS. 7B-7D illustrate a smaller bag (e.g., compared to that shown in FIG. 7A), a larger or longer bag may be used instead. Although example dimensions are indicated in FIGS. 6A and 6B, larger, smaller, or different ratios of dimensions can be used in other embodiments. As shown in FIG. 7A, the opening of the bag may be larger than the length or diameter of the ring portion of device 100 (e.g., so that the bag can wrap around the body of the device 100). In some embodiments, the opening of the bag is large enough such that the lip of the bag wraps around the ring portion of the device 100 as shown in FIG. 7B. In other embodiments, the opening of the bag is larger than the inner circumference of the ring portion of the device 100 but not large enough such that the lip of the bag wraps around the ring portion of the device 100. In such embodiments, once the cap is placed on the body, the lip of the bag would not be visible (e.g., caught or hidden inside the cavity and/or in between the top and bottom portions of the device 100).

After urinating, in embodiments, the upper fixture 1 is detached from the lower fixture 2. Subsequently, the bag 4 is removed from the lower fixture 2 and the plastic zipper is fastened to lock the bag 4. The used bag 4 is disposed and the upper fixture 1 and the lower fixture 2 can be retained in a place of the vehicle in the assembled state or an unassembled state for re-use.

The above-described configurations make it easy for the user to assemble (as shown in FIG. 7) and disassemble the device after use, and assemble again when storing the device for the compact purpose. Further, in embodiments, the device 100 is re-used while the bag 4 is disposable.

### Example Implementations (EIs)

Some enumerated example implementations (EIs) are provided in this section, without limitation.

EI 1: A portable urinary aid device comprising: an upper fixture having a ring shape; and a lower fixture configured to be engaged with the upper fixture, the lower fixture having a ring shape corresponding to the ring shape of the upper fixture and having at least one handle extending outwardly from one vertex of the ring shape thereof, wherein, when the upper fixture and the lower fixture are engaged, a hollow void is contained therein.

EI 2: The portable urinary aid device of EI 1, wherein the upper fixture has an oval shape that is laterally and longitudinally symmetrical, and the upper fixture has a concave shape when viewed laterally.

EI 3: The portable urinary aid device of EI 2, wherein the upper fixture comprises a channel configured to receive the lower fixture and edges configured to form an opening therebetween through which the lower fixture is inserted.

EI 4: The portable urinary aid device of EI 3, wherein the edges include an inner edge and an outer edge.

EI 5: The portable urinary aid device of EI 4, wherein the outer edge includes at least two dent portions provided at opposite locations on the outer periphery.

EI 6: The portable urinary aid device of EI 1, wherein the lower fixture has an oval shape that is laterally and longitudinally symmetrical, and the lower fixture has a concave shape when viewed laterally, so as to correspond to the upper fixture.

EI 7: The portable urinary aid device of EI 1, wherein the lower fixture has an upper surface and a lower surface, wherein the upper surface of the lower fixture includes an elevated portion disposed proximal to an inner edge of the lower surface.

EI 8: The portable urinary aid device of EI 1, wherein the handle extends diagonally downward from a lower surface of the lower fixture.

EI 9: The portable urinary aid device of EI 3, wherein the edges of the upper fixture are configured to engage with respective edges of the lower fixture.

EI 10: A device for securing a urine bag, the device comprising: an upper fixture having a ring shape; a lower fixture configured to be engaged with the upper fixture, the lower fixture having a ring shape corresponding to the ring shape of the upper fixture and having at least one handle coupled to one vertex one vertex of the ring shape thereof, wherein, when the upper fixture and the lower fixture are assembled, the assembly is configured to secure the urine bag between the upper and lower fixtures.

EI 11: The device of EI 10, wherein the upper fixture has an oval shape that is laterally and longitudinally symmetrical, and the upper fixture has a concave shape when viewed laterally.

EI 12: The device of EI 11, wherein an upper side of the upper fixture has a smooth surface and a lower side of the upper fixture has edges protruding outward such that a cross-section of the lower side has a U-shape.

EI 13: The device of EI 10, wherein the lower fixture has an oval shape that is laterally and longitudinally symmetrical, and the lower fixture has a concave shape when viewed laterally, so as to correspond to the upper fixture.

EI 14: The device of EI 10, wherein the lower fixture has an upper surface and a lower surface, wherein the upper surface of the lower fixture includes an elevated portion disposed proximal to an inner edge of the lower surface.

EI 15: The device of EI 10, wherein the handle extends diagonally downward from a lower surface of the lower fixture.

EI 16: The device of EI 12, wherein the edges of the upper fixture are configured to engage with respective edges of the lower fixture.

EI 17: A method of using the portable urinary aid device of EI 1, the method comprising: detaching the upper fixture from the lower fixture; unlocking a locking element of a urine bag to open an entrance of the urine bag; inserting the urine bag through the lower fixture and folding the urine bag over an upper surface of the lower fixture; and placing the upper fixture on the lower fixture to assemble the upper and lower fixtures such that the urine bag is immovably attached to the portable urinary aid device.

EI 18: The method of EI 17, further comprising, after the use of the portable urinary aid device: detaching the upper fixture from the lower fixture; removing the urine bag from the lower fixture; and securely locking the urine bag by the locking element.

EI 19: A method of using the device of EI 11, the method comprising: inserting a bag through a ring portion of the lower fixture such that a lip portion of the bag covers an upper surface of the lower fixture; and attaching the upper fixture to the lower fixture such that the bag is immovably fixed between the upper fixture and the lower fixture.

EI 20: The method of EI 19, further comprising: detaching the upper fixture from the lower fixture; removing the bag through the ring portion of the lower fixture; and securely locking the lip portion of the bag.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Thus, it is intended that the scope of the inventions herein disclosed should not be limited by the particular embodiments described above.

What is claimed is:

1. A portable urinary aid device comprising:
an upper fixture having a ring shape; and
a lower fixture configured to be engaged with the upper fixture, the lower fixture having a ring shape corresponding to the ring shape of the upper fixture and having at least one handle extending outwardly from one vertex of the ring shape thereof,
wherein, when the upper fixture and the lower fixture are engaged, a hollow void is contained therein.

2. The portable urinary aid device of claim 1, wherein the upper fixture has an oval shape that is laterally and longitudinally symmetrical, and the upper fixture has a concave shape when viewed laterally.

3. The portable urinary aid device of claim 2, wherein the upper fixture comprises a channel configured to receive the lower fixture and edges configured to form an opening therebetween through which the lower fixture is inserted.

4. The portable urinary aid device of claim 3, wherein the edges include an inner edge and an outer edge.

5. The portable urinary aid device of claim 4, wherein the outer edge includes at least two dent portions provided at opposite locations on the outer periphery.

6. The portable urinary aid device of claim 1, wherein the lower fixture has an oval shape that is laterally and longitudinally symmetrical, and the lower fixture has a concave shape when viewed laterally, so as to correspond to the upper fixture.

7. The portable urinary aid device of claim 1, wherein the lower fixture has an upper surface and a lower surface, wherein the upper surface of the lower fixture includes an elevated portion disposed proximal to an inner edge of the lower surface.

8. The portable urinary aid device of claim 1, wherein the handle extends diagonally downward from a lower surface of the lower fixture.

9. The portable urinary aid device of claim 3, wherein the edges of the upper fixture are configured to engage with respective edges of the lower fixture.

10. A device for securing a urine bag, the device comprising:
an upper fixture having a ring shape;
a lower fixture configured to be engaged with the upper fixture, the lower fixture having a ring shape corresponding to the ring shape of the upper fixture and having at least one handle coupled to one vertex one vertex of the ring shape thereof,
wherein, when the upper fixture and the lower fixture are assembled, the assembly is configured to secure the urine bag between the upper and lower fixtures.

11. The device of claim 10, wherein the upper fixture has an oval shape that is laterally and longitudinally symmetrical, and the upper fixture has a concave shape when viewed laterally.

12. The device of claim 11, wherein an upper side of the upper fixture has a smooth surface and a lower side of the upper fixture has edges protruding outward such that a cross-section of the lower side has a U-shape.

13. The device of claim 10, wherein the lower fixture has an oval shape that is laterally and longitudinally symmetrical, and the lower fixture has a concave shape when viewed laterally, so as to correspond to the upper fixture.

14. The device of claim 10, wherein the lower fixture has an upper surface and a lower surface, wherein the upper surface of the lower fixture includes an elevated portion disposed proximal to an inner edge of the lower surface.

15. The device of claim 10, wherein the handle extends diagonally downward from a lower surface of the lower fixture.

16. The device of claim 12, wherein the edges of the upper fixture are configured to engage with respective edges of the lower fixture.

17. A method of using the portable urinary aid device of claim 1, the method comprising:
detaching the upper fixture from the lower fixture;
unlocking a locking element of a urine bag to open an entrance of the urine bag;
inserting the urine bag through the lower fixture and folding the urine bag over an upper surface of the lower fixture; and
placing the upper fixture on the lower fixture to assemble the upper and lower fixtures such that the urine bag is immovably attached to the portable urinary aid device.

18. The method of claim 17, further comprising, after the use of the portable urinary aid device:
detaching the upper fixture from the lower fixture;
removing the urine bag from the lower fixture; and
securely locking the urine bag by the locking element.

19. A method of using the device of claim 11, the method comprising:
inserting a bag through a ring portion of the lower fixture such that a lip portion of the bag covers an upper surface of the lower fixture; and
attaching the upper fixture to the lower fixture such that the bag is immovably fixed between the upper fixture and the lower fixture.

20. The method of claim 19, further comprising:
detaching the upper fixture from the lower fixture;
removing the bag through the ring portion of the lower fixture; and
securely locking the lip portion of the bag.
